(19) United States
(12) Patent Application Publication
(10) Pub. No.: US 2005/0182366 A1
(43) Pub. Date: Aug. 18, 2005

(54) Method For Visual Output Verification

(75) Inventors: Marc Vogt, Rye, NH (US); Stephen Gemmell, Andover, MA (US)

Correspondence Address: INSULET CORPORATION, 9 Oak Park Drive, Bedford, MA, 01730, US

(73) Assignee: INSULET CORPORATION, Bedford, MA (US)

(21) Appl. No.: 10/908,089

(22) Filed: Apr. 27, 2005

Related U.S. Application Data
(63) Continuation-in-part of application No. 10/827,582, filed on Apr. 19, 2004.
(60) Provisional application No. 60/463,809, filed on Apr. 18, 2003.
(60) Provisional application No. 60/563,579, filed on Apr. 19, 2004.

(51) Int. Cl. A61M 37/00; A01N 31/00

(52) U.S. Cl. 604/131; 514/738

(57) ABSTRACT

A verification method including the steps of displaying a visual output in a first region of a user interface and displaying at least a portion of said visual output in at least a second region of said user interface, said at least a second region being different than said first region, wherein said first and said at least a second region are disposed on a single surface of a device.

### CROSS-REFERENCE TO RELATED APPLICATIONS

This application is a continuation-in-part of and claims the benefit of U.S. patent application Ser. No. 10/827582, which was filed on Apr. 19, 2004, and which claimed the benefit of U.S. Provisional Patent Application Nos. 60/463809, filed Apr. 18, 2003, and 60/563579, filed on Apr. 19, 2004, all of which are incorporated by reference herein.

### TECHNICAL FIELD

The present invention relates to user interfaces and more particularly, relates to method and apparatus for verifying the visual output of a user interface.

### BACKGROUND INFORMATION

Numerous devices include a user interface having a display device. Such devices visually display information to a user or operator. While there are a variety of different display devices, one of the most common display devices includes a screen such as a liquid crystal displays (LCDs). LCDs offer many advantages over traditional cathode ray tubes (CRTs) such as reduced size, reduced weight, and reduced power consumption. Most display devices, such as a LCDs and the like, use a plurality of segments or pixels that are manipulated to display the desired image. As used herein, segments and pixels are generally interchangeable.

For example, a simple and common LCD display segment arrangement 10 is shown in FIG. 1. By selectively manipulating the different segments or pixels 12, numerals can be displayed, for example the number “29”. A problem with such display arrangements 10, however, is that one or more segments or pixels 12 can malfunction resulting in the incorrect information being communicated to the user. This is particularly problematic with various medical apparatus where the display device 10 is used to display and input critical information.

Referring to FIG. 2, a user interface 14 for a medical device 16 is shown. For illustrative purposes only, and not a limitation of the present invention, the medical device 16 will be described as a remote controller 18 and an infusion pump 20 such as the remote controller 18 and infusion pump 20 described in U.S. Pat. Nos. 6,740,059 and 6,551,276, both of which are fully incorporated herein by reference as well as U.S. patent application Ser. No. 10/827582, which is also fully incorporated herein by reference.

The remote controller 18 includes a user interface 14 consisting of a display 22 having of a plurality of segments or pixels 12 and one or more input devices 24, such as one or more buttons or the like. Using the buttons 24, a user can enter data (such as input bolus amounts, delivery times, and the like) that is represented by the segments 12 and displayed on the display device 22.

If one or more segments or pixels 12 malfunctions, the incorrect information might be communicated. Referring specifically to FIGS. 1 and 3, should segment 26 malfunction, for example, the display may show “23” as shown in FIG. 3 and the user may incorrectly believe that he/she is administering a bolus amount of “23” when in fact they entered “29”. It goes without need of further explanation that this situation is obviously undesirable, particularly with respect to medical devices.

Accordingly, there exists a need for a method and apparatus to verify the correct operation of user interface display device. The method and apparatus should preferably quickly and easily alert a user to the malfunctioning of one or more segments or pixels of the display device. The method and apparatus should also preferably be inexpensive and not require extensive modification to implement.

It is important to note that the present invention is not intended to be limited to a system or method which must satisfy one or more of any stated objects or features of the invention. It is also important to note that the present invention is not limited to the preferred, exemplary, or primary embodiment(s) described herein. Modifications and substitutions by one of ordinary skill in the art are considered to be within the scope of the present invention, which is not to be limited except by the following claims.

### SUMMARY

In accordance with one aspect of the present invention, a verification method includes the acts of displaying a visual output in a first region of a user interface and displaying at least a portion of the visual output in at least a second region of the user interface. The second region is different than the first region, yet both the first and the second region are disposed on a single surface of a device. The device preferably includes a medical device such as an insulin infusion system including a remote control with the user interface disposed within the remote control.

The method optionally displays the visual output in the first region of the user interface while simultaneously displaying a portion of the visual output in the second region of the user interface. Alternatively, the visual output is displayed in the second region only while the data is being confirmed.

The method preferably includes the act of receiving data, wherein at least a portion of the data includes the visual output displayed in the first and the second region. The data may be received from a second, remote device or may be entered using one or more input devices disposed on the device. In response to the act of receiving data, the method preferably includes displaying the visual output in the first region of the user interface and verifying the visual output.

The act of verifying the visual output includes displaying in the second region at least a portion of the visual output displayed in the first region and receiving an acknowledgment of the visual output. The method preferably includes prompting a user to input an acknowledgment, preferably by displaying or communicating a verification message using the user interface.

The user interface optionally includes a first and at least a second separate display. The first region is disposed within the first display and the second region is disposed within the second display. Alternatively, the first and the second regions are disposed within a single display.

In accordance with another embodiment, the present invention features a verification system. The verification system includes a device having a housing and a user interface coupled to the housing. The user interface preferably includes at least one display having a first region and at least a second separate region both disposed on a first surface of the housing. The first and the second region are adapted to display a first and at least a second visual output, respectively. The second visual output includes at least a portion of the first visual output.

The user interface optionally includes at least one input device adapted to receive data, wherein the first and the second visual output each include at least a portion of the data. The input device preferably includes at least one button and/or one touch sensitive screen.

The display may include a first and at least a second separate display corresponding to the first and the second region, respectively. Alternatively, the display includes a single display including the first and the second region.

In accordance with yet a further embodiment, the present invention features a verification method comprising the acts of receiving data, displaying a first visual output in a first region of a user interface, displaying at least a second visual output in at least a second region of the user interface, and confirming that the first and the second visual output correspond. The first and the second visual output include at least a portion of the received data. The first and the second region are both disposed within a single device and the second region is different than the first region.

### BRIEF DESCRIPTION OF THE DRAWINGS

These and other features and advantages of the present invention will be better understood by reading the following detailed description, taken together with the drawings wherein:

FIG. 1 is a plan view of one embodiment of a plurality of segments forming a display of a user interface;

FIG. 2 is a plan view of the user interface shown in FIG. 1 in combination with a device;

FIG. 3 is a plan view of another embodiment of a plurality of segments forming a display of a user interface;

FIG. 4 is a plan view of one embodiment of the present invention in combination with an infusion system;

FIG. 5 is a flow chart of one embodiment of the verification method according to the present invention; and

FIG. 6 is a flow chart of another embodiment of the verification method according to the present invention.

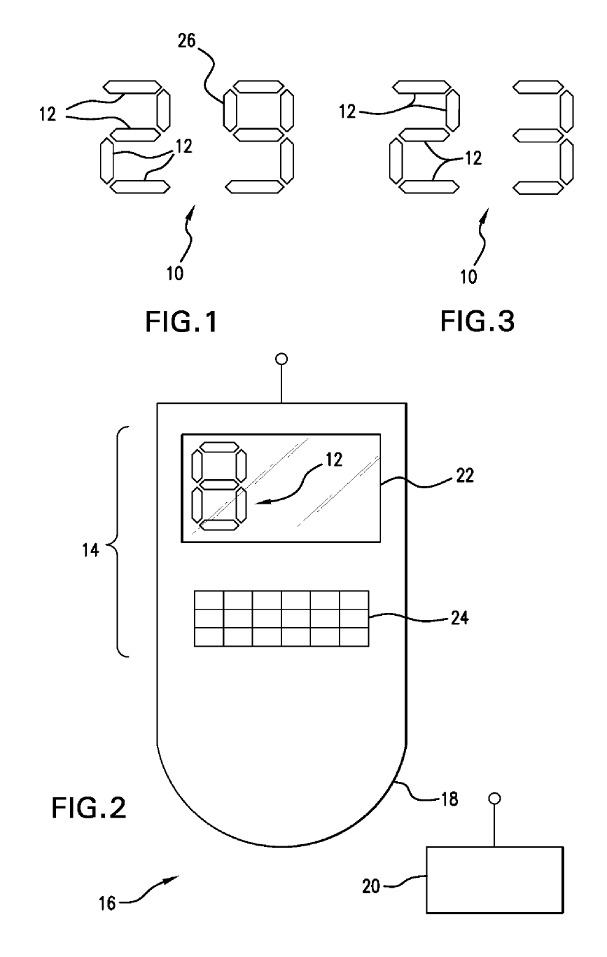

### DETAILED DESCRIPTION OF THE PREFERRED EMBODIMENTS

The present invention features a verification method and apparatus for verifying the correct operation of a display device such as a liquid crystal display (LCD) or the like. In the preferred embodiment, the method and apparatus preferably includes a medical device, though one skilled in the art will understand that the present invention is not limited to medical devices. While not an exhaustive list, in the exemplary embodiment the medical device includes an infusion system (either internal or external), a cardiac defibrillator, a neurostimulator, an x-ray machine, an EKG machine, a diagnostic device, a glucometer, a blood analyzer, an electrocautery device, an operating room table, a monitor, a laparoscopic controller, or the like, or a remote controller for use with one or more of the above-described medical devices. Additionally, the remote controller includes a dedicated remote controller as well as any other device capable of communicating with one or more medical devices such as, but not limited to, a cellular phone, a personal digital assistant, a computer (either a portable or desktop), a watch, and the like.

For illustrative purposes only, and not a limitation of the present invention unless otherwise specifically claimed, the present invention will be described with respect to an insulin delivery system as described in, for example, U.S. Pat. Nos. 6,740,059 and 6,551,276, both of which are fully incorporated herein by reference.

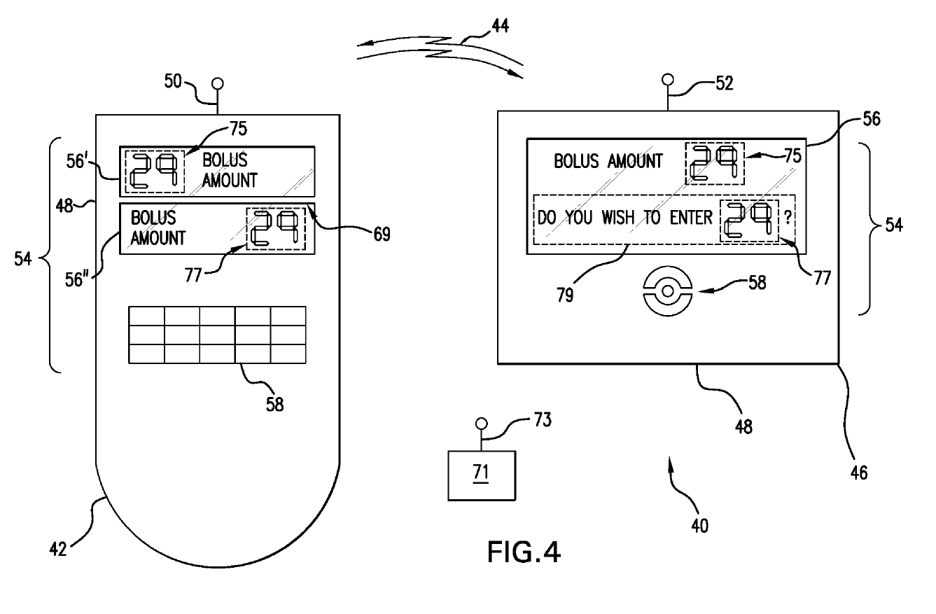

The insulin delivery system 40, FIG. 4, preferably includes a remote controller 42 operatively communicating (preferably wirelessly using RF signals 44) with an infusion pump 46 (either an internal or an external infusion pump 46). The remote controller 42 preferably includes a housing 48 made from a suitable plastic material and preferably includes a transceiver 50 that can transmit and/or receive signals 44 to/from a transceiver 52 of the infusion device 46 (or other device 71).

The remote controller 42 and/or the infusion device 46 includes a user interface 54 coupled to the housing 48. The user interface 54 includes one or more display devices 56 and optionally includes one or more inputs 58. The display 56 may include any display known to those skilled in the art, but preferably includes a liquid crystal display (LCD) or the like.

As used herein, the term “input” is intended to broadly include any device known to those skilled in the art for receiving data such as, but not limited to, one or more buttons, keys, mouse, trackballs, touch screens, voice communication software, data from another device (such as data received from either the remote controller 42, the infusion pump 46, or a remote database such as a server or the like).

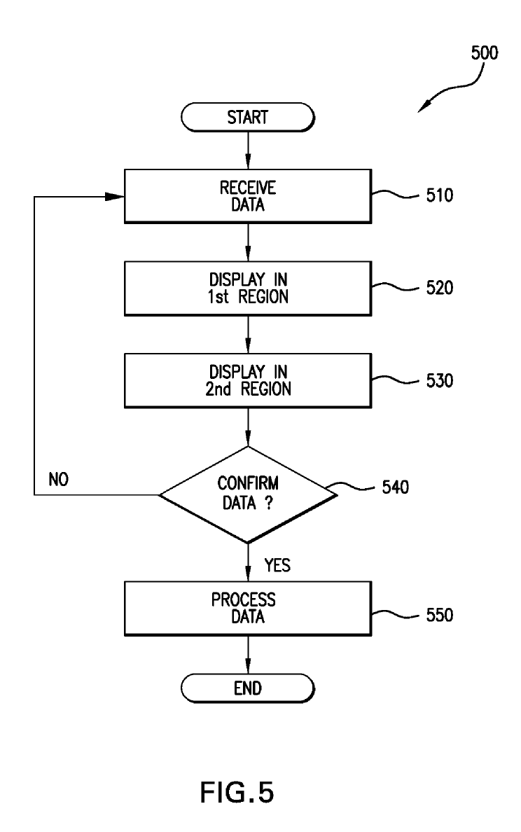

According to one embodiment, the present invention features a method 500, FIG. 5, and apparatus 40, FIG. 4, wherein data is received, act 510, by either the remote controller 42 (FIG. 4) and/or the infusion pump 46 (FIG. 4). In the preferred embodiment, a user inputs the data using the input device 58, FIG. 4. Alternatively, the data may be received from a remote device (such as the remote controller 42, infusion device 46, or a remote database 71, for example a server or the like) using the transceivers 50, 52, 73.

As used herein, the term “data” is intended to broadly include any information of significance to the user or device(s) or 42, 46, 71 including, but not limited to, input commands, amounts, times, instructions, and verification of commands, instructions, or the like.

Upon receipt/entry of the data, at least a portion of the data is displayed in a first region 75 of the display 56, act 520 (FIG. 5). In the preferred embodiment, act 520 occurs simultaneously with the receipt of the data, though there may be a slight delay. For illustrative purposes only, the act of receiving the data 510 includes a user entering a bolus amount of “29” via inputs 58.

Upon displaying the data in the first region 75 (act 520), at least a portion of the data displayed in the first region 75 is also simultaneously displayed in a second region 77 of the display 56, act 530. The first and the second region 75, 77 are preferably part of a single display 56 but are disposed within different areas of the display 56 such that the first and the second regions 75, 77 do not overlap. For example, the first the second regions 75, 77 may be offset to each other with respect to a longitudinal axis. Alternatively, or in addition to, the first and the second region 75, 77 may be offset with respect to a transverse axis.

Alternatively, the first and the second region 75, 77 may be disposed within a first and a separate, second display 56′, 56″ wherein both the first and the second display 56′, 56″ are both coupled to the same device 42, 46 and are both disposed within the same housing. The second display 56″ may be entirely separate from the first display 56′. Alternatively, the housing may include a divider or the like 69 that separates the display 56 into two distinct regions. In any event, it is important to note that the first and the second regions 75, 77 do not share any segments or pixels used to display critical data.

The user is asked to confirm or verify at least a portion of the data displayed in the first region 75 with at least a portion of the data displayed in the second region 77, act 540. For illustrative purposes only, the user is asked to confirm that the bolus amount “29” displayed within the first region 75 is the same as the bolus amount “29” displayed in the second region 77.

In the preferred embodiment, the act of confirming the data, 540, preferably includes presenting a verification message 79 to the user. The verification message 79 may include an audible tone, vibration, button, text message, or like. The act of confirming the data, 540, helps prevent incorrect data from being processed.

In the event of a malfunctioning segment/pixel or the like, the visual output of the first and the second display regions 75, 77 will not correspond. As a result, the present invention allows a user to verify the correct operation of the display 56 and alerts the user if a segment or pixel is malfunctioning in either region.

In the preferred embodiment, the user must then activate one or more input devices 58 to verify that the data is correct. Alternatively, if no action is taken to cancel or a like change the data within a predetermined amount of time, the data may be assumed to be accurate. In the event that the user does not confirm the data or takes some action to otherwise indicate that the data is incorrect, the process preferably starts over again and the data is not processed. Upon the data being confirmed, however, the data is then ready to processed, act 550 (for example, transmitting the bolus amount from the remote controller 42 to the infusion pump 46).

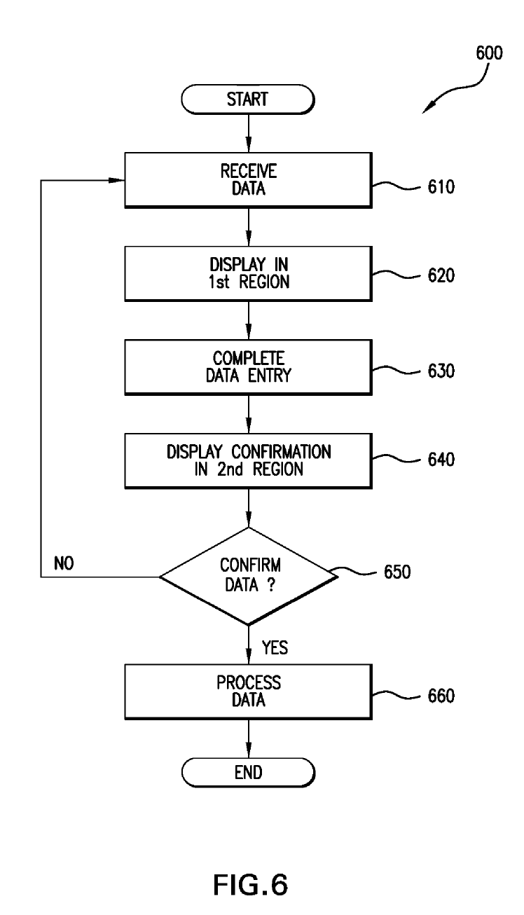

According to another embodiment, the present invention features a method 600, FIG. 6, and apparatus 40, FIG. 4, wherein data is received, act 610, displayed in the first region 75, act 620, as described above. Upon completion of the entering of the data, act 630, a confirmation message 79 is communicated, act 630. The confirmation message 79 includes any method or apparatus described herein, but must include displaying at least a portion of the data displayed in the first region 75 within the second region 77.

The user must then confirm the data, act 650, as described herein. In the event that the data is not confirmed, the process preferably starts over again. If the data is confirmed, the data is then processed, act 660.

Accordingly, the present invention includes a method and apparatus for verifying the correct operation of a visual output component of a user interface by displaying at least a portion of the visual output in at least two different regions of a display. As a result, the user can detect errors or malfunctions in one area of the display. Consequently, display failures or the like that could result in confusion or an incorrect data or command being enter can be avoided.

Those skilled in the art will recognize that the present invention is applicable to other types of devices (either medical or otherwise) having a user interface with a display. While not an exhaustive list, the present invention may also be used with a user interface of an external infusion pump, a cardiac defibrillator, a neurostimulator, an x-ray machine, an EKG machine, a diagnostic device, a glucometer, a blood analyzer, an electrocautery device, an operating room table, a monitor, a laparoscopic controller, a cellular phone, a personal digital assistant, a computer (either a portable or desktop), and the like.

As mentioned above, the present invention is not intended to be limited to a system or method which must satisfy one or more of any stated or implied object or feature of the invention and should not be limited to the preferred, exemplary, or primary embodiment(s) described herein. The foregoing description of a preferred embodiment of the invention has been presented for purposes of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as is suited to the particular use contemplated. All such modifications and variations are within the scope of the invention as determined by the claims when interpreted in accordance with breadth to which they are fairly, legally and equitably entitled.

1. A verification method comprising the acts of:
displaying a visual output in a first region of a user interface; and
displaying at least a portion of said visual output in at least a second region of said user interface, said at least a second region being different than said first region, wherein said first and said at least a second region are disposed on a single surface of a device.

2. The method as claimed in claim 1 wherein said device includes a medical device.

3. The method as claimed in claim 2 wherein said medical device includes an insulin delivery system.

4. The method as claimed in claim 3 wherein said insulin delivery system further includes a remote control and wherein said user interface is disposed within said remote control.

5. The method as claimed in claim 1 further including the act of displaying said visual output in said first region of said user interface while simultaneously displaying said at least a portion of said visual output in said at least a second region of said user interface.

6. The method as claimed in claim 1 further including the act of receiving data, wherein at least a portion said data includes said visual output displayed in said first and said second region.

7. The method as claimed in claim 6 further including the acts of:
in response to said act of receiving data, displaying said visual output in said first region of said user interface; and
verifying said at least a portion of said visual output comprising the acts of:
displaying said at least a portion of said visual output in said at least a second region of said user interface; and
receiving an acknowledgment of said at least a portion of said visual output.

8. The method as claimed in claim 7 wherein said act of receiving data into said device further includes receiving data from a second, remote device.

9. The method as claimed in claim 7 wherein said act of receiving data into said device further includes inputting user-supplied data via an input device disposed on said device.

10. The method as claimed in claim 7 wherein said act of receiving said acknowledgment further includes prompting a user to input an acknowledgment.

11. The method as claimed in claim 10 wherein said act of verifying said at least a portion of said visual output further includes the act of displaying a verification message within said user interface.

12. The method as claimed in claim 1 wherein said user interface includes a first and at least a second separate display, wherein said first region is disposed within said first display and said at least a second region is disposed within said at least a second display.

13. The method as claimed in claim 1 wherein said first and said at least a second regions are disposed within a single display.

14. A verification system comprising:
a device including a housing;
a user interface coupled to said housing and including at least one display having a first region and at least a second separate region both disposed on a first surface of said housing, said first and said at least a second region adapted to display a first and at least a second visual output, respectively, wherein said at least a second visual output includes at least a portion of said first visual output.

15. The verification system as claimed in claim 14 wherein said user interface further includes at least one input device adapted to receive data, wherein said first and said at least a second visual output each include at least a portion of said data.

16. The verification system as claimed in claim 15 wherein said at least one input device includes at least one button.

17. The verification system as claimed in claim 15 wherein said at least one input device includes at least one touch sensitive screen.

18. The verification system as claimed in claim 14 wherein said at least one display includes a first and at least a second display, wherein said first display includes said first region and said at least a second display includes said at least a second region.

19. The verification system as claimed in claim 14 wherein said at least one display includes a single display, wherein said single display includes said first and said at least a second region.

20. A verification method comprising the acts of:
receiving data;
displaying a first visual output in a first region of a user interface, said visual output including at least a portion of said data;
displaying at least a second visual output in at least a second region of said user interface, said second region being different than said first region, wherein said at least a second visual output includes at least a portion of said first visual output and wherein said first and said at least a second region are both disposed within a single device; and confirming that said first and said at least a second visual output correspond.
